(19) United States
(12) Patent Application Publication
(10) Pub. No.: US 2020/0138485 A1
(43) Pub. Date: May 7, 2020

(54) SI Joint Implant

(71) Applicants: Frank Kuwamura, San Antonio, TX (US); Thomas Zink, San Antonio, TX (US)

(72) Inventors: Frank Kuwamura, San Antonio, TX (US); Thomas Zink, San Antonio, TX (US)

(21) Appl. No.: 16/675,573

(22) Filed: Nov. 6, 2019

Related U.S. Application Data
(60) Provisional application No. 62/756,614, filed on Nov. 7, 2018.

(51) Int. Cl. A61B 17/70; A61F 2/30; A61B 17/86

(52) U.S. Cl. CPC A61B 17/7055; A61F 2/30988; A61B 17/8645; B33Y 80/00; A61F 2002/30029; A61F 2310/00023; A61F 2002/30995

(57) ABSTRACT

An improved sacroiliac (SI) joint implant device that comprises a compression screw and an additively manufactured bone matrix that promotes osteointegration between the device and a patient's bone. Specifically, the SI joint implant device will compress the patient's SI joint with the differential between the thread pitch of a distal portion of the device and a proximal portion of the device. As the SI joint implant device is advanced across the patient's SI joint, the smaller pitch on the proximal threads will compress the joint with every turn of the screw. The center section of the SI joint implant will also be sheathed in the additively manufactured bone matrix sleeve that has both the porosity and the structure to promote boney ingrowth.

### CROSS REFERENCE

This application claims priority from U.S. Provisional Patent Application Ser. No. 62/756,614 filed on Nov. 7, 2018, which is incorporated herein by reference.

### BACKGROUND

The present invention relates generally to a sacroiliac (SI) joint implant. Specifically, the sacroiliac joint implant comprises a compression screw and an additively manufactured bone matrix for osteointegration.

The sacroiliac joint is a large generally L-shaped synovial joint in the pelvis that connects the sacrum and the ilium of the pelvis, and is a generally strong, weight bearing joint on both sides of the pelvis. These joints are supposed to move and function together as a single unit, and provide shock absorption for the spine. Accordingly, just like other joints in the body, these joints have strong internal and external ligaments. However, as an individual ages, the joints' stability can change as ligaments become loose, and the joint surfaces lose their original orientation. Typically, when this occurs, a surgeon will take corrective action by fusing the joint with a compression screw.

Current compression screw designs for fusion of the SI joint are either comprised of metal compression screws, or a dowel rod made from osteo-integrative material. The metal compression screws are typically made from machined titanium alloy, or other medical/surgical grade materials. The dowel rod devices are typically machined rods plasma coated in titanium. Thus, existing technology either compresses the SI joint with a compression screw, or fuses the joint with porous titanium to promote osteointegration. Accordingly, the metal compression screws currently being utilized are pure machined titanium, and have great strength and compression properties but that do not allow for or promote osteointegration. In comparison, the dowel rod products currently being utilized permit and even promote osteointegration in the porous structures of the dowel rod, but do provide a high level of strength or always allow for sufficient compression of the joint.

Therefore, there is a long felt need in the art for a SI joint implant that combines the benefits of metal compression screws and surgical dowel rod products, without the associated limitations of said prior art products. More specifically, there is a long felt need in the art for an improved SI joint implant that provides a relatively high degree of strength and compression to the patient's joint, and that also promotes osteointegration between the implant device and the patient's bone. Finally, there is a long felt need in the art for an improved SI joint that is relatively easy to manufacture and use in a surgical setting, and that is both safe and effective.

The present invention discloses a sacroiliac joint implant device that comprises a surgical compression screw and an additively manufactured bone matrix sleeve component for promoting osteointegration between the device and the patient's bone. In this manner, the SI implant device combines the advantages of a surgical compression screw and the osteointegration capabilities of an additive titanium bone matrix into a single device. Specifically, the SI joint implant will compress the patient's SI joint with the differential between the distal and proximal thread pitch of the compression screw component. As the SI joint implant device is advanced across the joint, the smaller pitch on the proximal threads of the screw component will further compress the patient's SI joint with every turn of the compression screw. Further, the center section of the SI joint implant is preferably sheathed in an additively manufactured sleeve component that has a porosity and a structure that promotes boney ingrowth.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a sacroiliac joint implant device that comprises a surgical compression screw component and an additively manufactured bone matrix sleeve component that promotes osteointegration between the implant device and the patient's bone. Specifically, the SI joint implant device is preferably comprised of a surgical screw component that is further comprised of a distal portion and a proximal component that may be joined together to form a connection. An additively manufactured sleeve component can then be placed over the connection between the distal and proximal portions, and is useful for promoting osteointegration.

In another embodiment, the SI joint implant devices comprises a differential between the thread pitch of the distal and proximal portions, and the SI joint implant device will compress the SI joint of the patient with the differential between the distal and proximal thread pitches. More specifically, as the SI joint implant device is advanced across the patient's SI joint, the smaller pitch on the threads of the proximal portion will compress the joint with every turn of the screw. Further, the center section of the SI joint implant, namely where the proximal and distal portions are connected, will be sheathed in an additively manufactured sleeve component that has porosity and structure that promotes honey ingrowth between the device and the patient's bone.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a top perspective view of one embodiment of the sacroiliac (SI) joint implant device of the present invention in accordance with the disclosed architecture.

FIG. 2 illustrates a top partial perspective exploded view of the SI joint implant device of FIG. 1 in accordance with the disclosed architecture.

FIG. 3 illustrates a side perspective view of one embodiment of the additively manufactured sleeve component of the SI joint implant in accordance with the disclosed architecture.

FIG. 4 illustrates a top perspective view of one embodiment of the additively manufactured sleeve component of the SI joint implant in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention discloses an improved sacroiliac (SI) joint implant device that comprises a surgical compression screw component comprised of a distal portion and a proximal portion joined together to form a connection, and an additively manufactured bone matrix sleeve component for promoting osteointegration. More specifically, the SI joint implant device will compress a patient's SI joint with the differential between the thread pitches of the distal and the proximal portions of the surgical compression screw component. As the SI joint implant device is advanced across the SI joint of the patient, the relatively smaller pitch on the proximal portion threads will compress the joint with every turn of the screw. Additionally, the center section of the SI joint implant device, namely where the proximal portion of the compression screw component is connected with the distal portion, is preferably sheathed in an additively manufactured sleeve that has the porosity and structure to promote boney ingrowth between the device and the patient's bone.

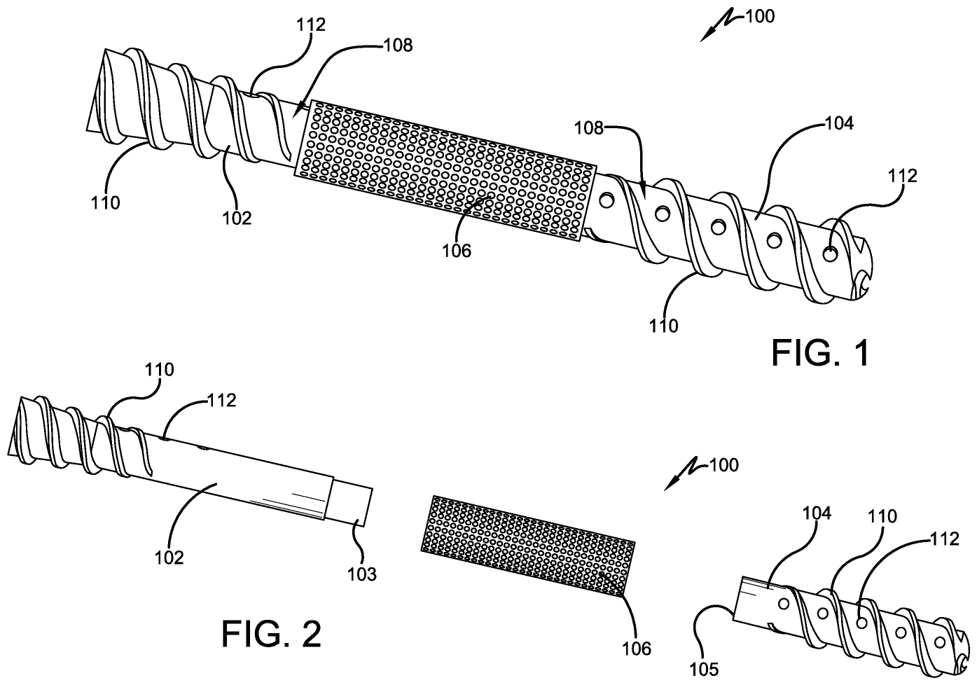

Referring initially to the drawings, FIG. 1 illustrates a top perspective view of one embodiment of the sacroiliac (SI) joint implant device 100 of the present invention in accordance with the disclosed architecture, and FIG. 2 illustrates a top partial perspective exploded view of the SI joint implant device 100. More specifically, SI joint implant device 100 is comprised of a cannulated proximal end component 102, a cannulated distal end component 104, and an osteo-integrative sleeve component 106. The SI joint implant device 100 can be any suitable size, shape, and configuration as is known in the art without affecting the overall concept of the invention. One of ordinary skill in the art will appreciate that the shape and size of the implant device 100 as shown in FIGS. 1 and 2 is for illustrative purposes only, and that many other shapes and sizes of the implant device 100 are well within the scope of the present disclosure. Although dimensions of the implant device 100 (i.e., length, width, and height) are important design parameters for good performance, the implant device 100 may be any shape or size that ensures optimal performance during use and/or that suits user need and/or preference.

Each of the proximal end component 102 and the distal end component 104 is preferably manufactured from a surgical grade metal, such as titanium, and specifically, Ti 6 Al 4 V-ELI. Nonetheless, it is contemplated that each of proximal end component 102 and the distal end component 104 can be manufactured from any other suitable surgical grade materials that may be known in the art.

Further, the proximal end component 102 is joined or connected to the distal end component 104 at their respective ends. Specifically, the proximal end component 102 and the distal end component 104 can be secured together at their ends via any securing means known in the art, such as threading, gluing, fasteners, friction fit, etc. For example, proximal end component 102 may further comprise a male portion 103 that can be received in a corresponding female opening 105 in the end of distal end component 104, as best shown in FIG. 2, or vice versa.

Once secured to one another, the proximal end component 102 and distal end component 104 forms a cannulated, generally cylindrical shaped screw component 108, but can be any other suitable shape as is known in the art. Specifically, the proximal end component 102 is preferably shaped such that the distal end component 104 is smaller in diameter than the proximal end component 102 of the SI joint implant device 100. Additionally, the screw component 108 comprises a plurality of screw threads 110 along the outer surface of the screw component 108, and the screw threads 110 preferably increase in diameter from the distal end component 104 to the proximal end component 102. More specifically, in a preferred embodiment of the present invention, the thread pitch of the screw threads 110 positioned along the outer surface of the proximal end component 102 is approximately 20% smaller than the thread pitch of the screw threads 110 positioned along the outer surface of the distal end component 104.

In a further preferred embodiment of the present invention, the screw component 108 also comprises a plurality of fenestrations 112 in the proximal end component 102 and/or the distal end component 104 that are in fluid communication with said cannulation. More specifically, the fenestrations 112 allow for bone promoting substances, such as demineralized bone matrix (DBM), allograft, cells, etc., to be extruded from the proximal end component 102 and/or the distal end component 104 into the surgical site. The fenestrations 112 are typically cylindrical in shape, but can be any other suitable shape as is known in the art for permitting bone promoting substances to pass therethrough.

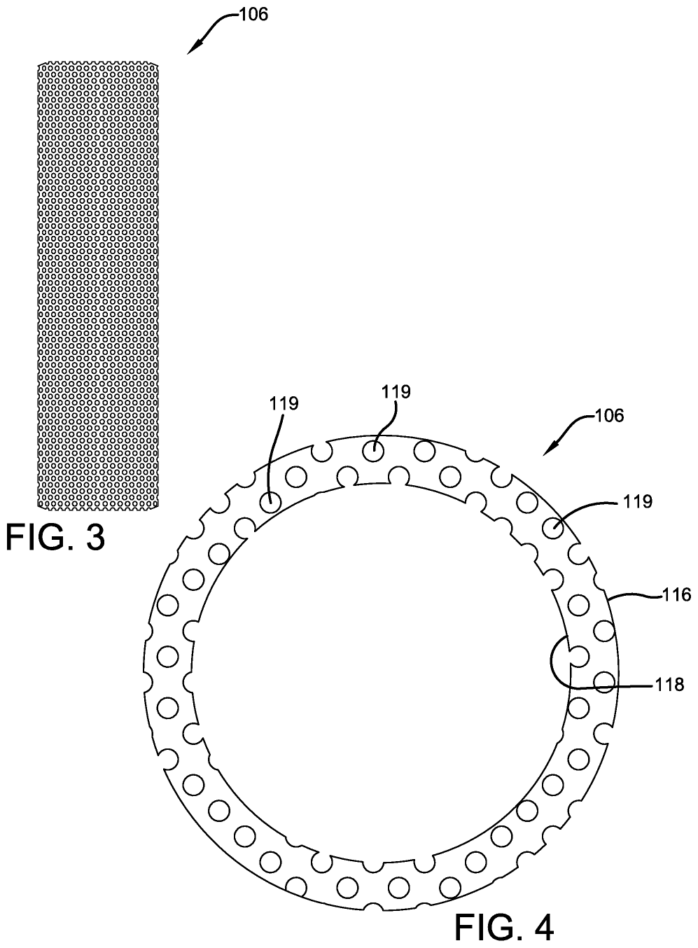

Once the proximal end component 102 and distal end component 104 are secured to one another to form a connection, an osteointegrative sleeve component 106 may then be secured over the connection between the proximal end component 102 and the distal end component 104. More specifically, the osteointegrative sleeve component 106 can be any suitable size, shape, and configuration as is known in the art without affecting the overall concept of the invention. One of ordinary skill in the art will appreciate that the shape, size and configuration of the osteointegrative sleeve component 106 shown in FIG. 3 is for illustrative purposes only, and that many other shapes, sizes and configurations of the osteointegrative sleeve component 106 are well within the scope of the present disclosure. Although dimensions of the osteointegrative sleeve component 106 (i.e., length, width, and height) are important design parameters for good performance, the osteointegrative sleeve component 106 may be any shape or size that ensures optimal performance during use or that suits user need and/or preference.

FIG. 3 illustrates a side perspective view of one embodiment of the osteointegrative sleeve component 106 of the SI joint implant device 100 in accordance with the disclosed architecture, and FIG. 4 illustrates a top perspective view of the osteointegrative sleeve component 106. In a preferred embodiment of the present invention, the osteointegrative sleeve component 106 is a generally tubular, bone growth promoting mesh structure comprised of additively printed titanium, though it is contemplated that other surgical grade materials capable of promoting bone growth may also be used. More specifically, the osteointegrative sleeve component 106 is comprised of an outer surface 116, an inner surface 118, and a plurality of porous openings 119 positioned therebetween. The osteointegrative sleeve component 106 is preferably manufactured using additive manufacturing (AM) techniques and grown as a single unitary part, such as via 3-d printing or other such techniques.

Having described but a few of the potential embodiments of the SI joint implant device 100 of the present invention, its use will now be described in general terms. Generally, in use, a patient will be placed on a table in a surgical setting in a lateral position. The SI joint will then be mapped with an X-ray or other fluoroscopic device. A wire is then placed into the SI joint medially to enable the surgeon to see trajectory and location, and dilators are used to open the surgical site. The SI joint implant device 100 is then threaded into the patient's SI joint, thereby pulling or compressing it together. Specifically, the SI joint implant device 100 will compress the patient's SI joint with the differential between the thread pitch of the distal end component 104 and the thread pitch of the proximal end component 102. More specifically, as the implant device 100 is advanced across the patient's SI joint, the smaller pitch on the threads of the proximal end component 104 will compress the SI joint of the patient with every turn of the implant device 100. Additional SI joint implant devices 100 may then be placed linearly along the patient's joint to fixate the joint rotationally.

After the SI joint implant device or devices 100 have been successfully implanted in the patient's SI joint, a bone growth promoting substance, such as DBM, allograft, cells, etc., may then be pushed or injected into the cannulation of the SI joint implant 100, and extruded from the fenestrations 112 in the implant device 100 and into the surgical site to promote osteointegration of the implant device 100 with the patient's bone. Additionally, the presence of sleeve component 106 and its various porous openings 119 over the connection formed by the mating of distal end component 104 and proximal end component 102 also promotes osteointegration of the implant device 100 with the patient's bone.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sacroiliac joint implant comprising:
a screw component; and
an osteointegrative sleeve component.

2. The sacroiliac joint implant of claim 1, wherein the screw component is comprised of a proximal end component and a distal end component.

3. The sacroiliac joint implant of claim 2, wherein the proximal end component is fixedly attached to the distal end component.

4. The sacroiliac joint implant of claim 2, wherein each of the proximal end component and the distal end component further comprises a plurality of external threads.

5. The sacroiliac joint implant of claim 4, wherein the plurality of external threads of the proximal end component have a first pitch, and the plurality of external threads of the distal end component have a second pitch.

6. The sacroiliac joint implant of claim 5, wherein the first pitch is different than the second pitch.

7. The sacroiliac joint implant of claim 2, wherein each of the proximal end component and the distal end component further comprises a cannulation and a plurality of fenestrations.

8. The sacroiliac joint implant of claim 7, wherein the cannulation is in fluid communication with the plurality of fenestrations.

9. A sacroiliac joint implant for installation in a patient's joint and comprising:
a proximal end component and a distal end component attached to said proximal end component; and
an osteointegrative sleeve component partially positioned over each of the proximal end component and the distal end component.

10. The sacroiliac joint implant of claim 9, wherein the proximal end component and the distal end component compress the patient's joint.

11. The sacroiliac joint implant of claim 9, wherein the osteointegrative sleeve component comprises a porosity and structure that promotes boney ingrowth.

12. The sacroiliac joint implant of claim 9, wherein each of the proximal end component and the distal end component further comprises a plurality of external threads.

13. The sacroiliac joint implant of claim 12, wherein the plurality of external threads of the proximal end component have a first pitch, and the plurality of external threads of the distal end component have a second pitch.

14. The sacroiliac joint implant of claim 13, wherein the first pitch is different than the second pitch.

15. The sacroiliac joint implant of claim 9, wherein each of the proximal end component and the distal end component further comprises a cannulation and a plurality of fenestrations.

16. The sacroiliac joint implant of claim 15, wherein the cannulation is in fluid communication with the plurality of fenestrations.

17. A joint implant comprising:
a proximal end component comprised of a titanium alloy;
a distal end component comprised of the titanium alloy; and
an additively manufactured osteointegrative sleeve component.

18. The joint implant of claim 17, wherein each of the proximal end component and the distal end component further comprises a plurality of external threads, and further wherein the plurality of external threads of the proximal end component have a first pitch, and the plurality of external threads of the distal end component have a second pitch.

19. The joint implant of claim 18, wherein the first pitch is different than the second pitch.

20. The joint implant of claim 17, wherein each of the proximal end component and the distal end component further comprises a cannulation in fluid communication with a plurality of fenestrations.
